(19) United States
(12) Patent Application Publication
(10) Pub. No.: US 2010/0125294 A1
(43) Pub. Date: May 20, 2010

(54) LANCET ASSEMBLY

(76) Inventor: Shinichi YASUI, Ehime (JP)

Correspondence Address: WENDEROTH, LIND & PONACK L.L.P., 1030 15th Street, N.W., Suite 400 East, Washington, DC, 20005-1503, US

(21) Appl. No.: 12/272,086

(22) Filed: Nov. 17, 2008

(51) Int. Cl. A61B 17/34

(52) U.S. Cl. 606/182

(57) ABSTRACT

A lancet assembly 100 comprises a lancet 1 which has a puncture element at its one end, a cap 6 which covers the puncture element, a holder 40 which movably holds the lancet 1, a pressing lever 30 which drives the lancet 1, and an ejector 20 which urges the lancet 1 and drives the lancet 1 in accordance with the operation of the pressing lever 30, and the lancet assembly 100 is configured such that the cap 6 is engaged with the pressing lever 30 and moved in the ejection direction of the lancet 1 to be separated from the lancet 1.

### FIELD OF THE INVENTION

The present invention generally relates to a lancet assembly for collecting a small amount of blood by wounding a patient's skin, and more particularly, to a lancet assembly which is improved so as to facilitate its use.

### BACKGROUND OF THE INVENTION

While a lancet assembly has previously been subjected to sterilization when manufactured, it is required that the sterilized state of the lancet is maintained in a good condition so as not to be contaminated by the surrounding environment up to its puncture operation being performed. To realize that end, the lancet assembly is configured such that a puncture element is covered with such as a resin membrane, i.e., a cap, and the cap is removed to expose the puncture element before performing the puncture operation.

As disclosed in Patent Document 1 (Brochure of International Publication No. 96/16599), a conventional lancet assembly comprises a lancet and an ejector which are housed in a holder, and it is configured such that the lancet is formed of a resin, holds a puncture element inside, and has a front end which is covered with a resin cap that is adjoined by any of various methods such as adhesive bonding, welding, and integral molding. In addition, a notch is formed at the boundary between the lancet and the cap, and a puncture operator holds the holder with one hand while holding an end of the cap with the other hand and pulls them apart from each other before the puncture operation, whereby a breakage of the resin occurs at the portion of the notch, and thus the cap can be removed to expose the puncture element, thereby enabling puncture.

In the conventional configuration, however, the puncture operator is required to carry out a process of removing the cap from the lancet assembly using his both hands and thereafter performing a puncture operation by his one hand while holding the patient's site to be punctured with his the other hand. Accordingly, the both hands of the puncture operator are temporarily employed for the works of preparing the lancet assembly, resulting in an excessive action being required for the puncture operation, or resulting in an interruption in the flow of successive medical operations due to the both hands of the puncture operator being temporarily restrained.

### SUMMARY OF THE INVENTION

The present invention is made to solve the above-described problems and has for its object to provide a lancet assembly which enables the puncture operator to perform the puncture operation without performing the excessive operation, that is, the lancet assembly preparation work for removing the cap.

Other objects and advantages of the invention will become apparent from the detailed description that follows. The detailed description and specific embodiments described are provided only for illustration since various additions and modifications within the scope of the invention will be apparent to those of skill in the art from the detailed description.

According to a first aspect of the present invention, there is provided a lancet assembly including a lancet which has a puncture element at its one end, a cap which covers the puncture element, a holder which movably holds the lancet, a pressing lever which drives the lancet in its ejection direction, and an ejector which urges the lancet and drives the lancet in accordance with the operation of the pressing lever, wherein when the ejector is operated, the pressing lever and the cap are engaged with each other and thereby the cap is moved in the ejection direction to be separated from the lancet.

According to a second aspect of the present invention, in the lancet assembly according to the first aspect, the lancet comprises a resin molded body which houses the puncture element, the puncture element is coated with a resin at its exposed portion, a notch is provided at a predetermined portion of the resin, which predetermined portion is to be broken, and the lancet assembly is separable into two parts including the cap which covers the exposed portion and the lancet which holds the puncture element.

According to a third aspect of the present invention, in the lancet assembly according to the first aspect, the ejector ejects the lancet, and pulls the lancet back to a predetermined position after the lancet was ejected.

According to a fourth aspect of the present invention, in the lancet assembly according to the first aspect, the holder includes a guide member which makes the lancet linearly move, a guide member which makes the pressing lever move, and a trigger which restrains the lancet until the pressing lever reaches a predetermined position and triggers the lancet to eject after the reaching.

According to a fifth aspect of the present invention, in the lancet assembly according to the first aspect, after the pressing lever being moved in the lancet ejection direction is contacted with the cap of the lancet which is restrained by the holder, the cap is moved in the lancet ejection direction thereby to break the notch to expose the puncture element.

According to a sixth aspect of the present invention, in the lancet assembly according to the second aspect, after the pressing lever being moved in the lancet ejection direction is contacted with the cap of the lancet which is restrained by the holder, the cap is moved in the lancet ejection direction thereby to break the notch to expose the puncture element.

According to a seventh aspect of the present invention, in the lancet assembly according to the first aspect, after the cap is separated from the lancet due to the movement of the pressing lever in the lancet ejection direction, the cap is ejected outward from the inner center position of the holder.

According to an eighth aspect of the present invention, in the lancet assembly according to the first aspect, after the cap is separated from the lancet due to the movement of the pressing lever in the lancet ejection direction, the cap is moved along a guide provided in the holder due to further movement of the pressing lever to be departed from the lancet ejection path.

According to a ninth aspect of the present invention, in the lancet assembly according to the fourth aspect, after the pressing lever having a part for releasing the trigger starts to move and thereby the trigger is moved to a second position, the lancet which has been restrained by the trigger is ejected.

According to a tenth aspect of the present invention, in the lancet assembly according to the first aspect, a first pressing lever and a second pressing lever are provided as the pressing lever, the first pressing lever moves the cap to separate the cap from the lancet, and the second pressing lever starts its movement after the first pressing lever is operated, and ejects the lancet.

According to an eleventh aspect of the present invention, in the lancet assembly according to the first aspect, the ejector comprises a spring element which is previously compressed or expanded, and is engaged with the holder at its one end and with an end portion of the lancet at its the other end.

According to a twelfth aspect of the present invention, in the lancet assembly according to the third aspect, the ejector comprises a spring element which is previously compressed or expanded, and is engaged with the holder at its one end and with an end portion of the lancet at its the other end.

According to a thirteenth aspect of the present invention, in the lancet assembly according to the first aspect, the ejector comprises a resin or metal spring element which is, in its unloaded condition, engaged and attached to the pressing lever at its one end and to the lancet at its the other end, and is compressed or expanded in accordance with the movement of the pressing lever.

According to a fourteenth aspect of the present invention, in the lancet assembly according to the third aspect, the ejector comprises a resin or metal spring element which is, in its unloaded condition, engaged and attached to the pressing lever at its one end and to the lancet at its the other end, and is compressed or expanded in accordance with the movement of the pressing lever.

According to a fifteenth aspect of the present invention, in the lancet assembly according to the tenth aspect, the ejector comprises a spring element which is connected to the pressing lever at its one end and is engaged with the lancet at its the other end.

According to a sixteenth aspect of the present invention, in the lancet assembly according to the first aspect, the puncture element is needle-shaped.

According to a seventeenth aspect of the present invention, in the lancet assembly according to the second aspect, the puncture element is needle-shaped.

According to an eighteenth aspect of the present invention, in the lancet assembly according to the first aspect, the puncture element is blade-shaped.

According to a nineteenth aspect of the present invention, in the lancet assembly according to the second aspect, the puncture element is blade-shaped.

According to a twelfth aspect of the present invention, in the lancet assembly according to the first aspect, after the lancet is incorporated in the holder, the resin of the notch is cut or melted to be removed such that only a thin resin layer is formed around the puncture element, and the thin resin layer is broken by pushing the pressing lever to separate the cap from the lancet.

### EFFECT OF THE INVENTION

According to the lancet assembly of the present invention, the operation thereof can be performed with one hand throughout the entirety of the puncture operation process, thereby facilitating the puncture operation.

### BRIEF DESCRIPTION OF THE DRAWINGS

FIG. 1(a) is a perspective view of a lancet assembly in its initial state before operation, according to a first embodiment of the present invention.

FIG. 1(b) is a front view of a holder which constitutes the lancet assembly of the first embodiment.

FIG. 1(c) is a front elevational view illustrating the state where a lancet is set in the holder which constitutes the lancet assembly of the first embodiment.

FIG. 1(d) is a front view illustrating the state where a pressing lever is further set in the holder which constitutes the lancet assembly of the first embodiment.

FIG. 1(e) is a front view illustrating the state where a latch member and an ejector are further set in the holder which constitutes the lancet assembly of the first embodiment.

FIG. 2 is a perspective view illustrating a major part of the configuration of the lancet 1 in the lancet assembly 100 of the first embodiment.

FIG. 3 is a schematic front view illustrating the lancet assembly 100 of the first embodiment in the state where a lid 41 is removed.

FIG. 4 is a front view illustrating the lancet assembly 100 of the first embodiment in the state where the puncture operation advances from the state of FIG. 3, and the pressing lever 30 moves the cap 6 in the ejection direction.

FIG. 5 is a front view illustrating the lancet assembly 100 of the first embodiment in the state where the puncture operation advances from the state of FIG. 4, and the pressing lever 30 moves the cap 6 to a place which is apart from the ejection path of the lancet 1.

FIG. 6 is a front view illustrating the lancet assembly 100 of the first embodiment in the state where the puncture operation advances from the state of FIG. 5, and the pressing lever 30 deforms a part of the holder 40 to release the lancet 1 from restriction.

FIG. 7 is a front view illustrating the lancet assembly 100 of the first embodiment in the state where the released lancet 1 is ejected, and a puncture element 5 protrudes from the holder 40.

FIG. 8 is a front view illustrating the lancet assembly 100 of the first embodiment in the state where the entire puncture operation is completed.

FIG. 9 is a schematic front view illustrating the construction of a lancet assembly 200 of a second embodiment of the present invention, which is obtained by changing the ejector element in the lancet assembly 100 of the first embodiment to an ejector element of another configuration.

FIG. 10 is a schematic front view illustrating the construction of a lancet assembly 300 of a third embodiment of the present invention, which is obtained by changing an ejector 60 in the lancet assembly 200 of the second embodiment to an elector element of still another configuration.

FIG. 11 is a schematic front view illustrating a lancet assembly 400 of a fourth embodiment of the present invention.

FIG. 12 is a schematic perspective view illustrating a lancet assembly 500 of a fifth embodiment of the present invention in the state where the cap 6 is separated from the lancet 1.

FIG. 13 is a schematic perspective view illustrating the lancet assembly 500 of the fifth embodiment in the state where the cap 6 is separated from the lancet 1.

FIG. 14 is a schematic perspective view illustrating the lancet 1 from which the cap 6 is not yet separated, in the lancet assembly 500 of the fifth embodiment.

FIG. 15 is a front view illustrating an enlarged major part of the lancet assembly 500 of the fifth embodiment shown in FIG. 14.

### DETAILED DESCRIPTION OF THE PREFERRED EMBODIMENTS

Hereinafter, preferred embodiments of lancet assemblies of the present invention will be described in detail with reference to the drawings.

### Embodiment 1

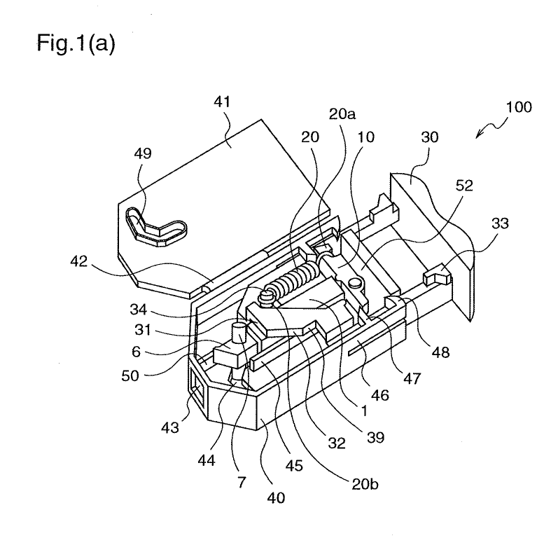

FIG. 1(a) shows a perspective view of a lancet assembly 100 in its initial state before operation, according to a first embodiment of the present invention.

FIGS. 1(b) to 1(e) show the process steps for manufacturing the lancet assembly 100 shown in FIG. 1(a).

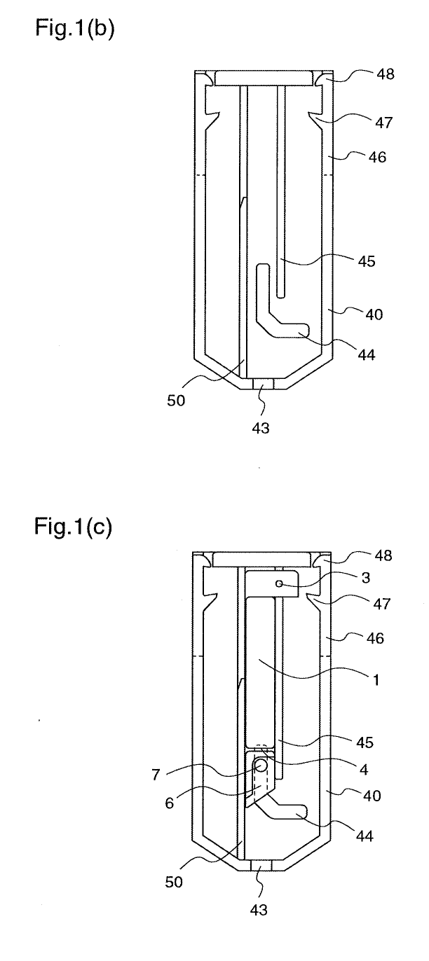

FIG. 2 is a perspective view showing a main part of the lancet assembly 100 to clarify the configuration of a lancet 1 shown in FIG. 1(a).

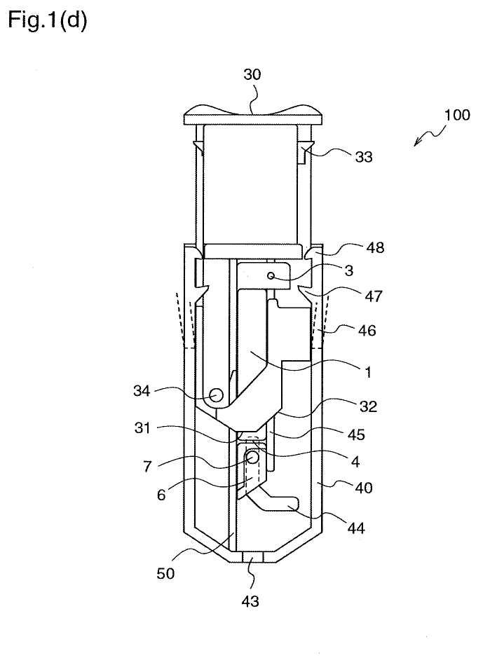

FIG. 3 is a schematic front view of the lancet assembly 100 in the state of FIG. 1(a), showing the state where a lid 41 is removed to clarify the internal structure.

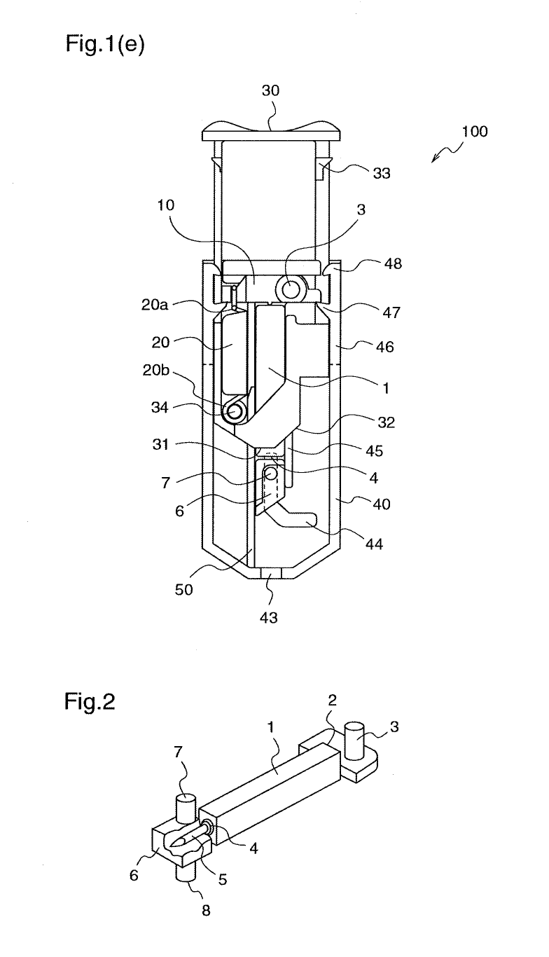

FIG. 4 shows a front view of the lancet assembly 100 in the state where the puncture operation advances from FIG. 3, and a pressing lever 30 moves a cap 6 in the ejection direction.

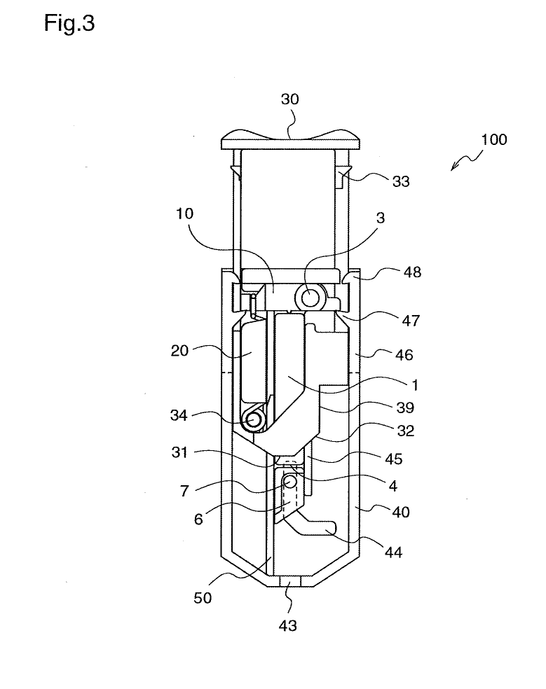

FIG. 5 shows a front view of the lancet assembly 100 in the state where the puncture operation advances from FIG. 4, and the pressing lever 30 moves the cap 6 to a place which is apart from the ejection path of the lancet 1.

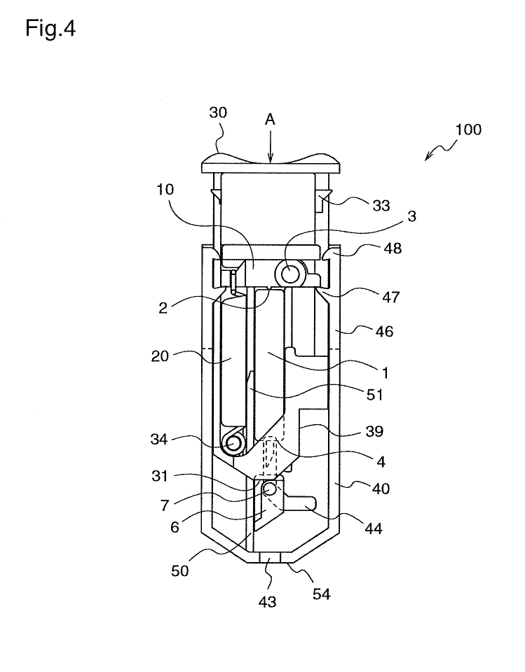

FIG. 6 shows a front view of the lancet assembly 100 in the state where the puncture operation advances from FIG. 5, and the pressing lever 30 deforms an elastic plate 46 of a holder 40 to release the lancet 1 from restriction.

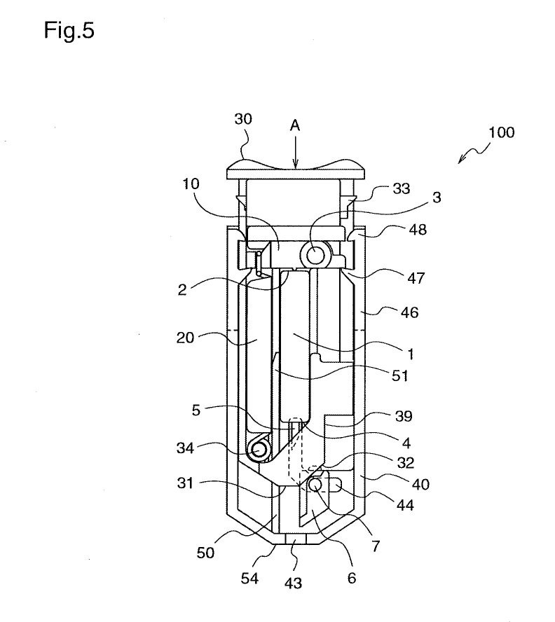

FIG. 7 shows a front view of the lancet assembly 100 in the state where the released lancet 1 is ejected, and a puncture element 5 protrudes from the holder 40.

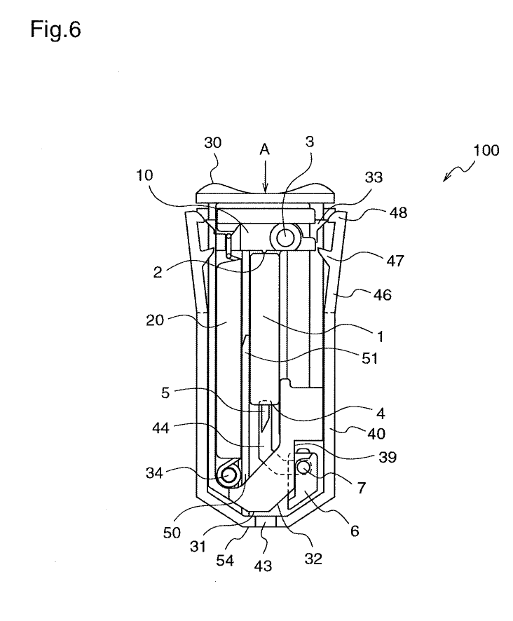

FIG. 8 shows a front view of the lancet assembly 100 in the state where the whole puncture operation is completed.

Next, the configuration of the lancet assembly 100 according to the first embodiment of the present invention will be described with reference to FIG. 1(a).

In FIG. 1(a), the holder 40 holds the pressing lever 30 linearly slidably, and includes guide ribs 45 and 50 which moves the lancet 1 linearly in the ejection direction, an elastic plate 46 and a trigger 47 provided on the elastic plate 46, which contact a locking member 10 that is engaged with the lancet 1 rotatably around a rotation axis 3 to restrict the lancet 1 from being ejected, a release claw which contacts a release cam 33 provided at a part of the pressing lever 30 to elastically deform the elastic plate 46 of the holder 40, and a lid 41 which is molded integrally with the holder 40 via a hinge 42.

An end 20a of an ejector 20 which supplies a force for ejecting the lancet 1 is engaged with the latch member 10 while the other end 20b of the ejector 20 is engaged with a pin-shaped member 34 of the pressing lever 30, and the pressing lever 30 is moved along the ejection direction by the operation of the puncture operator, and simultaneously, the ejector 20 is expanded to store a force for ejection.

When the pressing lever 30 starts to move, its anterior edge 31 contacts a guide shaft 7 of the lancet 1, and the cap 6 is moved along a guide groove 44 of the holder 40 and a guide groove 49 of the lid 41. The specific configuration of the lancet 1 will be described with reference to FIG. 2.

The lancet 1 is manufactured by integral molding of a resin having the puncture element 5 inside. The lancet 1 comprises the cap 6 which covers a front end portion of the puncture element 5, cylindrical guide shafts 7 and 8 formed on both the front and back sides of the cap 6, a notch 4 provided at the boundary between the cap 6 and the lancet 1, a cylindrical rotary shaft 3 which rotatably engages the latch member 10 with the lancet 1, and a rear end face 2 which contacts the latch member 10 at a predetermined position when the latch member 10 is rotated with the force of the ejector 20, thereby to serve as a stopper for the rotation of the latch member 10, and the lancet 1 and the cap 6 are maintained to be tightly attached to the puncture element 5.

The lancet assembly 100 housing the lancet 1 is manufactured as follows. As shown in FIG. 1(b), the holder 40 has the guide ribs 45 and 50 which are formed in parallel with each other at the bottom of the holder 40 along its longitudinal direction, and an approximately L-shaped guide groove 44 is formed in the vicinity of a front end of the guide rib 45 while an opening 43 is formed at one side of the holder 40 which is positioned on an extended line from the guide rib 45.

The lancet 1 is set from above between the guide ribs 45 and 50 of the holder 40 having such configuration, thereby obtaining the state shown in FIG. 1(c).

Next, after the elastic plate 46 of the holder 40 is expanded outward to set the pressing lever 30, the expanded elastic plate 46 is released to be restored to the original position by its elasticity, thereby obtaining the state shown in FIG. 1(d). This pressing lever 30 is formed integrally with the release cam 33, the pin-shaped member 34, and the like.

Next, the pressing lever 30 is slightly pushed downward, and the latch member 10 is set in a space between the trigger 47 and the release claw 48. At this time, the rotary shaft 3 of the lancet 1 is fitted into the opening of the latch member 10. Thereafter, an end of the ejector 20 is engaged with the latch member 10 while the other end thereof is engaged with the pin-shaped member 34, thereby obtaining the state shown in FIG. 1(e). Thereafter, the lid 41 shown in FIG. 1(a) is closed. At this time, the lid 41 is fixed to the holder 40 by engaging a claw or the like (not shown) provided on the lid 41 or by adhering the lid 41 to the holder 40 with an adhesive agent or the like.

Through a series of works described above, the disposal lancet assembly 100 is completed.

Hereinafter, the operation of the lancet assembly 100 configured as described above will be described with reference to FIGS. 3 to 8.

In the pre-operation state shown in FIG. 3, since the anterior edge 31 of the pressing lever 30 is separated from the upper end of the cap 6 by a predetermined distance, the pressing lever 30 will not contact the cap 6 even if the pressing lever 30 is moved by an accidental external force, and thus the cap 6 is prevented from being apart from the puncture element 5 by false operation. Further, the ejector 20 maintains its most contracted state, and the force given to between the pressing lever 30 and the latch member 10 is small. This prevents the respective members of the lancet assembly which are formed of resin in many cases from being creep-deformed, which is a kind of deterioration with time, when the lancet assembly is stored for long periods.

FIG. 4 shows the state where the puncture operator starts to push the pressing lever 30 in the direction of arrow A, and more specifically, it shows the state where the pressing lever 30 receiving the force of arrow A is moved in the ejection direction of the lancet 1, the anterior edge 31 of the pressing lever 30 contacts the cap 6, the force of arrow A is directly given to the cap 6 and thereby a breakage occurs in the notch 4, the cap 6 is separated from the lancet 1, and the cap 6 is moved in the ejection direction of the lancet 1 (toward lower side of the lancet 1 in the figure) along the puncture element 5. At this time, the movement of the cap 6 mainly goes along the puncture element 5, and the restriction on the movement direction by the engagements of the guide grooves 44 and 49 with the guide shafts 7 and 8 is subsidiary. Further, the ejector 20 starts to expand as the pin-shaped member 34 moves toward the front end of the lancet 1 in accordance with the movement of the pressing lever 30, and stores an ejection force.

FIG. 5 shows the state where the pressing lever 30 further moves in the ejection direction, and the pressing lever 30 makes the cap 6 continue to move, with the face at which the cap 6 contacts the pressing lever 30 being changed from the anterior edge 31 of the pressing lever 30 to the cam face 32 that makes an angle of about 45° with respect to the anterior edge 31. At this time, the movement direction of the cap 6 is determined by only the guide grooves 44 and 49 and the guide shafts 7 and 8. This movement direction is changed to a direction diagonal (about 45°) to the ejection direction in accordance with the shapes of the guide grooves 44 and 40. When the pressing lever 30 further moves, the cap 6 reaches the terminal ends of the guide grooves 44 and 49 to stop the movement by the cam face 39 parallel to the ejection direction of the pressing lever 40. The terminal ends of the guide grooves 44 and 49 are formed in a direction perpendicular to the ejection direction. The position where the cap stops its movement is a position where the cap would not impede the forward movement of the lancet 1 when the lancet 1 is ejected.

By further pushing the pressing lever 30, as shown in FIG. 6, the release claw 48 at the inner side of the front end of the elastic plate 46 of the holder 40 contacts the release cam 33 of the pressing lever 30 to elastically deform the elastic plate 46 toward the outside of the holder 40, and the trigger 47 which similarly moves outward due to the deformation of the elastic plate 46 is separated from the latch member 10. Thereby, the ejector 20 is contracted, and the lancet 1 engaged with the latch member 10 is ejected along the guide ribs 45 and 50, and then the exposed puncture element 5 protrudes outward from the outer edge 54 of the holder 40 which touches the skin (not shown) through the opening 43 by a predetermined length as shown in FIG. 7.

At this time, the lancet 1 tends to move by its inertia until its rear end contacts the inner face at the opening 43 side of the holder 40. In the vicinity of the terminal end of this movement route, the latch member 10 contacts the stopper 51 provided at a predetermined position in the holder 40, and the latch member 10 and the stopper 51 go into the sliding relation while rotating in the clockwise direction in FIG. 7 around the rotary shaft 3, thereby to move the lancet 1 until it contacts the inner face of the holder 40, and simultaneously, expansion of the ejector 20 is started, and thereby the lancet 1 which has been moved to the predetermined position is pulled back in the direction reverse to the injection direction.

In many cases, the means for ejecting and pulling back the lancet 1 is simply configured so as to be realized by the balance between the inertia of the lancet that is directly attached to a single compression spring and the stretching force of the spring, as described in Patent Document 1. However, when utilizing the reciprocal motion of the single compression spring as described above, the reciprocal motion does not stop by only a single reciprocation, but several times of reciprocal motions with attenuation, i.e., howling, undesirably occur. Since, due to this howling, the lancet moves in the ejection direction plural times and thereby the puncture element projects from the holder plural times, the patient is subjected to plural times of punctures. Such punctures cause an increase of pain, and give a fear of puncture to the patient.

The configuration of the first embodiment resolves this problem. That is, when the lancet 1 is pulled back in the direction reverse to the ejection direction, a frictional force which is caused by rotation and sliding between the latch member 10 and the stopper 51 has an effect of a damper to attenuate the force of the ejector 20, and therefore, the ejector 20 cannot store a force enough to move the lancet 1 in the ejection direction again. Therefore, the lancet 1 never moves in the ejection direction again, and the puncture element 5 never protrudes from the holder 40.

Then, as shown in FIG. 8, the ejector 20 is contracted, and the lancet 1 terminates its movement at a position where the ejector 20 and the lancet 1 are balanced with the stopper 51 in the center. This position is a position the human hand cannot touch, in which the puncture element 5 moves inward by a predetermined position from the outer edge of the holder 40.

Further, in this state, the release cam 33 of the pressing lever 30 and the release claw 48 of the holder 40 are engaged with each other, and thus the release cam 33 and the release claw 48 serve as a safety device. That is, by engaging the release cam 33 with the release claw 48, the pressing lever 30 is pulled back to the position where it was unused to make the lancet assembly 100 unusable again. Accordingly, the lancet assembly 100 is disposed after blood collection was once performed using the same.

As described above, according to the lancet assembly 100 of this first embodiment, by only pushing the pressing lever 30 in the ejection direction, the cap 6 is separated and moved from the lancet 1, and the lancet 1 is ejected to a predetermined site of the patient to complete the puncture operation. Therefore, the puncture operator can easily perform the puncture operation by only his actions of holding the patient's site with his one hand and simultaneously taking the lancet assembly with his the other hand and then pushing the pressing lever 30 with his fingers.

### Embodiment 2

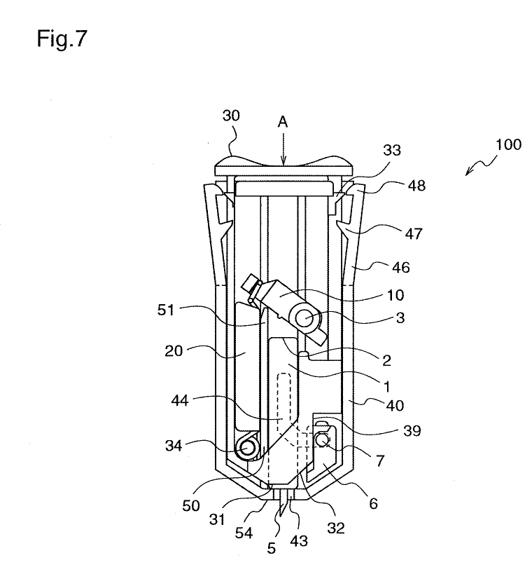

FIG. 9 is a schematic front view illustrating the configuration of a lancet assembly 200 of a second embodiment, which is obtained by changing the ejector elements in the lancet assembly 100 of the first embodiment.

With reference to FIG. 9, the lancet assembly 200 of this second embodiment includes an ejector 60 which stores a force by contraction, typified by a compression spring, and one end 60a of the ejector 60 is engaged with a convex portion for positioning which is formed on a rear face 52 of the holder 40 while the other end 60b thereof is inserted into a long hole 11 formed in the lancet 1.

The configuration of this second embodiment is different from the first embodiment in that the ejector 60 is incorporated in the holder 40 while being previously contracted to store a force for ejecting the lancet 1. Thereby, after the lancet 1 is ejected with the stored ejection force, the reaction force is received by the rear face 52 of the holder 40 and the position of the long hole 11 of the lancet 1 which are the both ends of the ejector 60, and therefore, the lancet assembly 100 can have a sufficient strength which prevents deformation over long periods, and further, the latch member 10 and the stopper 51 used in the first embodiment can be dispensed with.

The operation of the lancet assembly 200 of this second embodiment is identical to the first embodiment until the pressing lever 30 starts to move and the lancet 1 is ejected, and it is different from the first embodiment only in that the length of the ejector 60 when it is unloaded is utilized to pull the puncture element 5 projected from the outer edge of the holder 40 back into the holder 40.

More specifically, by setting the length of the ejector 60 when it is unloaded to a length shorter than a difference obtained by subtracting the distance between the position of the long hole 11 of the lancet 1 and the front end of the puncture element 5 in the lancet 1 from the distance between the rear face 52 of the holder 40 and the outer edge 54 of the holder 40, the front end of the puncture element 5 is housed inside the holder 40 most of the time.

As described above, according to the lancet assembly 200 of this second embodiment, the ejector which stores a force by its contraction is previously incorporated in the holder in its contracted state, and the lancet is pulled back into the holder utilizing that the ejector returns to the length in its unloaded condition after the lancet is ejected. Therefore, the latch member 10 of the first embodiment for returning the puncture element back into the holder after it is protruded from the holder is dispensed with, and thus a similar puncture operation to that of the first embodiment can be realized with a simpler configuration.

### Embodiment 3

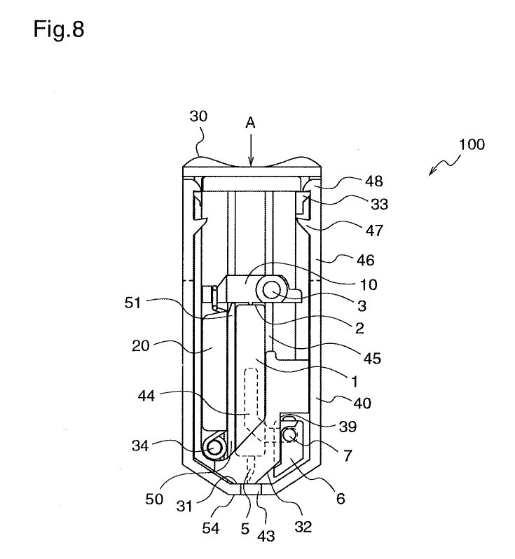

FIG. 10 is a schematic front view illustrating the configuration of a lancet assembly 300 according to the third embodiment of the present invention, which is obtained by replacing the ejector 60 in the lancet assembly 200 of the second embodiment shown in FIG. 9 with another element.

With reference to FIG. 10, the pressing lever 30 has a elastically deformable curvature portion 35, and its front end 38 is engaged with the lancet 1, and thereby the curvature portion 35 increases its elastic deformation amount with the movement of the pressing lever 30. Therefore, when the release cam 33 of the pressing lever 30 releases the release claw 48, the deformation amount becomes maximum, and thus a predetermined ejection force can be stored.

As described above, according to the lancet assembly 300 of this third embodiment, the pressing lever is provided with the elastically deformable curvature portion, and the lancet is pulled back into the holder by utilizing that this curvature portion returns to the length in its unloaded condition like the ejector 60 after the lancet is ejected. Therefore, the ejector is dispensed with, and a similar puncture operation can be carried out with the configuration that is simpler than that of the second embodiment shown in FIG. 9.

### Embodiment 4

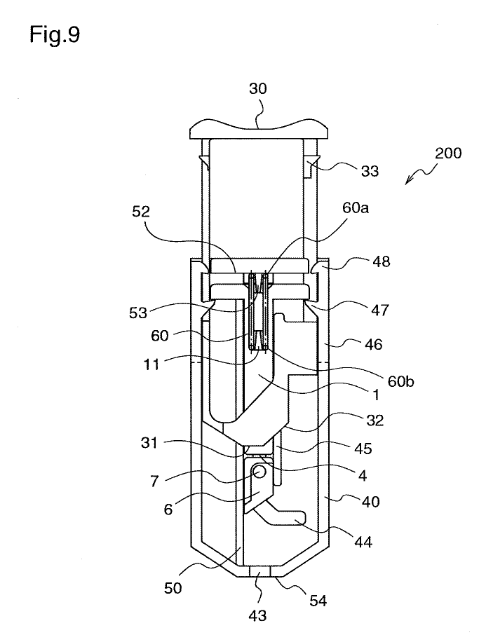

FIG. 11 shows a schematic front view of a lancet assembly 400 according to a fourth embodiment of the present invention.

The lancet assembly 400 of this fourth embodiment is different from the lancet assembly 200 of the second embodiment shown in FIG. 9 in that the pressing lever 30 which is a single member in the second embodiment is separated into two members including a first pressing lever 36 for separating and moving the cap 6 from the lancet 1 and a second pressing lever 37 for ejecting the lancet 1.

As described above, according to the lancet assembly 400 of this fourth embodiment, initially the cap 6 of the lancet 1 is removed by operating the first pressing lever 36 to expose the puncture element 5, and thereafter, the lancet 1 is made to be ejected at a desired timing by the second pressing lever 37. Therefore, the timing of removing the cap 6 and the timing of performing ejection of the lancet 1 can be made different according to the puncture operator's will, thereby enabling the puncture operation more adaptable to the puncture operator's will.

Moreover, in the lancet assembly 400 of this fourth embodiment, it is not necessary for the puncture operation to rehold the lancet assembly between the operation of the first pressing lever 30 and the operation of the second pressing lever 37, and the puncture operation can be carried out as a series of operations, thereby providing a simple puncture operation also in this fourth embodiment.

While in the first to fourth embodiments the cap removed from the lancet is stored in the holder, it may be ejected from the holder by, for example, providing an opening on the side of the holder.

### Embodiment 5

While in the first to fourth embodiments the lancet assembly which enables the operator to remove the cap from the lancet and eject the lancet by his one-hand operation is described, a lancet assembly of a fifth embodiment enables reliable separation of the cap by operating the pressing lever, and avoids that the cap is removed due to an external force or the like which is applied during transfer and thus the puncture element cannot be maintained in the sterile condition.

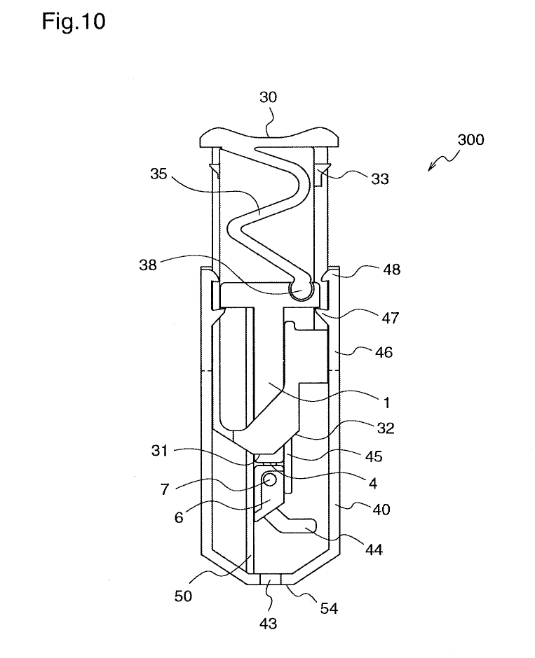

FIG. 12 is a schematic perspective view illustrating a lancet assembly 500 of this fifth embodiment in the state where the cap 6 is separated from the lancet 1.

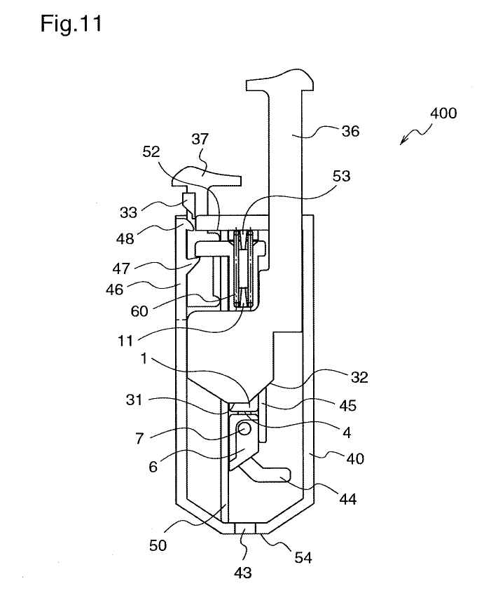

FIG. 13 is a schematic perspective view illustrating the state where the cap 6 is separated from the lancet 1 in the fifth embodiment.

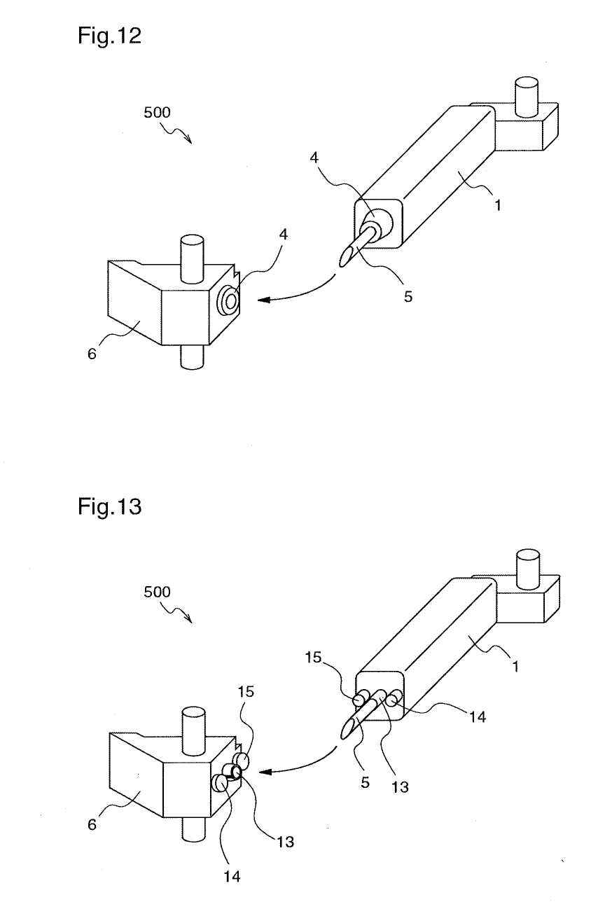

FIG. 14 is a schematic front view illustrating the lancet 1 before the cap 6 is separated from the lancet 1 in this fifth embodiment.

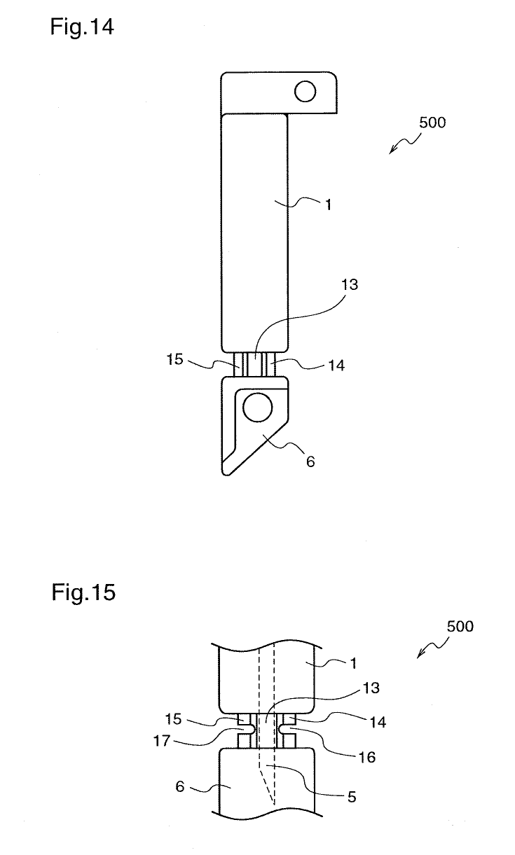

FIG. 15 is a front view illustrating an enlarged major part of the lancet 1.

As shown in FIG. 12, the notch 4 is resin-molded in the state where it connects the lancet 1 and the cap 6 and covers the puncture element 5 inside, and the notch 4 is broken and separated by operating the pressing lever 30 (not shown).

In order to break the notch 4 with a light pressing force during operation, the thickness of the notch 4 covering the puncture element 5 is desired to be thinner. In such case, however, the notch 4 might be broken with receiving an accidental external force when the lancet 1 is handled during its incorporation work or transfer, and the cap 6 is undesirably separated to expose the puncture element 5, and thus the sterile condition of the puncture element 5 cannot be maintained.

In the fifth embodiment shown in FIGS. 13 to 15, the lancet 1 has a thin-wall notch 13 obtained by reducing the thickness of the resin layer covering the puncture element 5, and reinforcing ribs 14 and 15 which are continuous to the thin-wall notch 13 to avoid the above-mentioned breakage. When the notch 13 is thus reinforced, handling of the lancet 1 during its assembly and transfer is identical to that described in the first embodiment. When manufacturing the lancet assembly 500, the lancet 1 is incorporated in the holder 40, and slits 16 and 17 are formed in the reinforcing ribs 14 and 15 as shown in FIG. 15 under the state where no external force is applied to the lancet 1.

As a method for forming the slits, a well-known cutting method such as press cutting, thermal melting, supersonic dissolution, or laser dissolution is adopted, and the slits 16 and 17 are not formed in the thin-walled notch 13. Thereby, the sterile condition of the puncture element 5 is maintained, and the pressing force required for separating the cap 6 is reduced, and thus the cap can be reliably separated by operating the pressing lever.

The lancet 1 can be used independently without being incorporated in the lancet assembly 500. In this case, although the advantage that the cap can be removed with one hand is not obtained, the cap is prevented from being removed due to an external force during transfer or the like, and the cap can be easily removed as required by previously giving a simple processing to the reinforcing ribs.

As described above, according to the lancet assembly 500 of this fifth embodiment, the thickness of the notch which connects the lancet and the cap is reduced, the reinforcing ribs are provided to prevent the cap from dropping off due to an external force which occurs during transfer or the like, and the slits are formed into the reinforcing ribs when no external force is applied to the lancet in such as assembling the lancet assembly. Therefore, it is possible to avoid that the cap is removed due to an external force which occurs during transfer or the like and thereby the sterile condition of the puncture element 5 cannot be maintained, and further, it is possible to easily perform removal of the cap when the lancet is used.

### APPLICABILITY IN INDUSTRY

As described above, according to the lancet assembly of the present invention, the puncture operator can complete the puncture operation with only his one hand throughout the entirety of the puncture operation process while avoiding that his both hands are temporarily employed for the works of preparing the lancet assembly to remove the cap for maintaining the sterile condition and thereby the flow of the successive medical works is undesirably interrupted, and thus a lancet assembly which can facilitate the puncture operation can be obtained.

1. A lancet assembly including a lancet which has a puncture element at its one end, a cap which covers the puncture element, a holder which movably holds the lancet, a pressing lever which drives the lancet in its ejection direction, and an ejector which urges the lancet and drives the lancet in accordance with the operation of the pressing lever, wherein
when the ejector is operated, the pressing lever and the cap are engaged with each other and thereby the cap is moved in the ejection direction to be separated from the lancet.

2. A lancet assembly as defined in claim 1, wherein
said lancet comprises a resin molded body which houses the puncture element, and
said puncture element is coated with a resin at its exposed portion, and a notch is provided at a predetermined portion of the resin, said predetermined portion being to be broken,
said lancet assembly being separable into two parts including the cap which covers the exposed portion and the lancet which holds the puncture element.

3. A lancet assembly as defined in claim 1, wherein
said ejector ejects the lancet, and pulls the lancet back to a predetermined position after the lancet was ejected.

4. A lancet assembly as defined in claim 1, wherein
said holder includes a guide member which makes the lancet linearly move, a guide member which makes the pressing lever move, and a trigger which restrains the lancet until the pressing lever reaches a predetermined position and triggers the lancet to eject after the reaching.

5. A lancet assembly as defined in claim 1, wherein
after the pressing lever being moved in the lancet ejection direction is contacted with the cap of the lancet which is restrained by the holder, the cap is moved in the lancet ejection direction thereby to break the notch to expose the puncture element.

6. A lancet assembly as defined in claim 2, wherein
after the pressing lever being moved in the lancet ejection direction is contacted with the cap of the lancet which is restrained by the holder, the cap is moved in the lancet ejection direction thereby to break the notch to expose the puncture element.

7. A lancet assembly as defined in claim 1, wherein
after the cap is separated from the lancet due to the movement of the pressing lever in the lancet ejection direction, the cap is ejected outward from the inner center position of the holder.

8. A lancet assembly as defined in claim 1, wherein
after the cap is separated from the lancet due to the movement of the pressing lever in the lancet ejection direction, the cap is moved along a guide provided in the holder due to further movement of the pressing lever to be departed from the lancet ejection path.

9. A lancet assembly as defined in claim 4, wherein
after the pressing lever having a part for releasing the trigger starts to move and thereby the trigger is moved to a second position, the lancet which has been restrained by the trigger is ejected.

10. A lancet assembly as defined in claim 1, wherein
a first pressing lever and a second pressing lever are provided as the pressing lever,
said first pressing lever moving the cap to separate the cap from the lancet, and
said second pressing lever starting its movement after the first pressing lever is operated, and ejecting the lancet.

11. A lancet assembly as defined in claim 1, wherein
said ejector comprises a spring element which is previously compressed or expanded, and is engaged with the holder at its one end and with an end portion of the lancet at its the other end.

12. A lancet assembly as defined in claim 3, wherein
said ejector comprises a spring element which is previously compressed or expanded, and is engaged with the holder at its one end and with an end portion of the lancet at its the other end.

13. A lancet assembly as defined in claim 1, wherein
said ejector comprises a resin or metal spring element which is, in its unloaded condition, engaged and attached to the pressing lever at its one end and to the lancet at its the other end, and is compressed or expanded in accordance with the movement of the pressing lever.

14. A lancet assembly as defined in claim 3, wherein
said ejector comprises a resin or metal spring element which is, in its unloaded condition, engaged and attached to the pressing lever at its one end and to the lancet at its the other end, and is compressed or expanded in accordance with the movement of the pressing lever.

15. A lancet assembly as defined in claim 10, wherein
said ejector comprises a spring element which is connected to the pressing lever at its one end and is engaged with the lancet at its the other end.

16. A lancet assembly as defined in claim 1, wherein said puncture element is needle-shaped.

17. A lancet assembly as defined in claim 2, wherein said puncture element is needle-shaped.

18. A lancet assembly as defined in claim 1, wherein said puncture element is blade-shaped.

19. A lancet assembly as defined in claim 2, wherein said puncture element is blade-shaped.

20. A lancet assembly as defined in claim 1, wherein
after the lancet is incorporated in the holder, the resin of the notch is cut or melted to be removed such that only a thin resin layer is formed around the puncture element, and the thin resin layer is broken by pushing the pressing lever to separate the cap from the lancet.
